(19) United States
(12) Patent Application Publication
(10) Pub. No.: US 2013/0169948 A1
(43) Pub. Date: Jul. 4, 2013

(54) METHOD FOR RAPID IMAGING OF BIOLOGIC FLUID SAMPLES

(71) Applicant: Abbott Point of Care, Inc., Princeton, NJ (US)

(72) Inventors: Min A. Xie, Plainsboro, NJ (US); David Herzog, Warrington, PA (US); John Verrant, Solebury, PA (US)

(73) Assignee: Abbott Point of Care, Inc., Princeton, NJ (US)

(21) Appl. No.: 13/729,887

(22) Filed: Dec. 28, 2012

Related U.S. Application Data
(60) Provisional application No. 61/581,851, filed on Dec. 30, 2011.
(60) Provisional application No. 61/594,136, filed on Feb. 2, 2012.

(51) Int. Cl. G01N 33/49

(52) U.S. Cl. CPC G01N 33/49
USPC 356/40

(57) ABSTRACT

A method for analyzing a biologic fluid sample is provided. The method includes the steps of: a) disposing the biologic fluid sample within a chamber; b) imaging the biologic fluid sample at a first resolution, and producing first image signals representative of a low resolution image of the sample; c) analyzing the first image signals to identify one or more first characteristics of the sample, and determining a position of each first characteristic within the chamber using a map of the chamber; d) imaging a portion of the biologic fluid sample at a second resolution and producing second image signals, which portion of the sample is determined using the first characteristics and the map, and wherein the second resolution is greater than the first resolution; and e) analyzing the biologic fluid sample using the second image signals.

The present application is entitled to the benefit of and incorporates by reference essential subject matter disclosed in the following U.S. Provisional Patent Applications: Ser. Nos. 61/581,851, filed Dec. 30, 2011; and 61/594,136, filed Feb. 2, 2012.

### BACKGROUND OF THE INVENTION

1. Technical Field

The present invention generally relates to methods for imaging a biologic fluid sample, and more specifically relates to methods and apparatuses for imaging a biologic fluid sample at more than one resolution and in some instances less than the entire sample.

2. Background Information

Historically, biologic fluid samples such as whole blood, urine, cerebrospinal fluid, body cavity fluids, etc., have had their particulate or cellular contents evaluated by smearing a small undiluted amount of the fluid on a slide and evaluating that smear under a manually operated microscope. Reasonable results are attainable using these techniques, but they rely heavily upon the technician's experience and technique. These techniques are also labor-intensive and thus not practically feasible for commercial laboratory applications.

Automated apparatuses for analyzing biologic fluid samples are known, including some that are adapted to image a sample of biologic fluid quiescently residing within a chamber. Automated analysis devices can produce results that are as accurate as manual examination methods in a substantially reduced period of time. Nonetheless, the speed at which automated devices operate can be significantly limited by high resolution imaging. High resolution imaging produces substantial volumes of electronic data that must be processed by the apparatus. It would be desirable to provide an automated device and methodology that reduced the time required to consistently provide accurate results.

### SUMMARY OF THE DISCLOSURE

According to an aspect of the present invention, a method for analyzing a biologic fluid sample is provided. The method includes the steps of: a) disposing the biologic fluid sample within a chamber adapted to quiescently hold the biologic fluid sample; b) imaging the biologic fluid sample at a first resolution, and producing first image signals representative of a low resolution image of the sample; c) analyzing the first image signals to identify one or more first characteristics of the sample, and determining a position of each first characteristic within the chamber using a map of the chamber; d) imaging a portion of the biologic fluid sample at a second resolution and producing second image signals representative of a high resolution image of the sample, which portion of the biologic fluid sample is determined using the one or more first characteristics and the map, and wherein the second resolution is greater than the first resolution; and e) analyzing the biologic fluid sample using the second image signals.

According to another aspect of the present invention, an apparatus for analyzing a biologic fluid sample quiescently disposed within an analysis chamber is provided. The apparatus includes an objective lens assembly, at least one image dissector, and a processor. The objective lens assembly is operable to image the biologic fluid sample at a first resolution and a second resolution, which second resolution is greater than the first resolution. The processor is adapted to: a) analyze first image signals produced by the image dissector with the objective lens at the first resolution; b) identify one or more first characteristics of the sample; c) determine a position of each first characteristic within the chamber using a map of the chamber; d) create an image of a portion of the biologic fluid sample at the second resolution and to produce second image signals representative thereof, which portion of the biologic fluid sample is determined using the one or more first characteristics and the map; and e) analyze the biologic fluid sample using the second image signals.

According to another aspect of the present invention, a method for imaging a biologic fluid sample is provided. The method includes the steps of: a) disposing the biologic fluid sample within a chamber adapted to quiescently hold the biologic fluid sample, which sample fills an area within the chamber; b) mapping the chamber, which map defines a plurality of grid squares, each grid square having an area; and c) imaging portions of the biologic fluid sample, each image portion aligned with a different grid square of the map, and each image portion having an area, and which image portions together form a collective image of the sample residing within the chamber, and wherein the collective area of the image portions has an area that is less than the area filled by the sample residing within the chamber.

The present method and advantages associated therewith will become apparent in light of the detailed description of the invention provided below, and as illustrated in the accompanying drawings.

### BRIEF DESCRIPTION OF THE DRAWINGS

FIG. 1 is a perspective view of a biological fluid sample analysis cartridge.

FIG. 2 is an exploded, perspective view of the biological fluid sample analysis cartridge shown in FIG. 1.

FIG. 3 is a planar view of a tray holding an analysis chamber.

FIG. 4 is a sectional view of an analysis chamber.

FIG. 5 is a diagrammatic view of an analysis device.

FIG. 6 is a flow diagram illustrating the present invention imaging methodology.

FIG. 7A is a diagrammatic illustration of a map applied to a chamber residing in an X-Y plane, including image portions centered in each grid square of the map.

FIG. 7B is a diagrammatic illustration of a map applied to a chamber residing in an X-Y plane, including image portions randomly disposed in each grid square of the map.

### DETAILED DESCRIPTION OF THE INVENTION

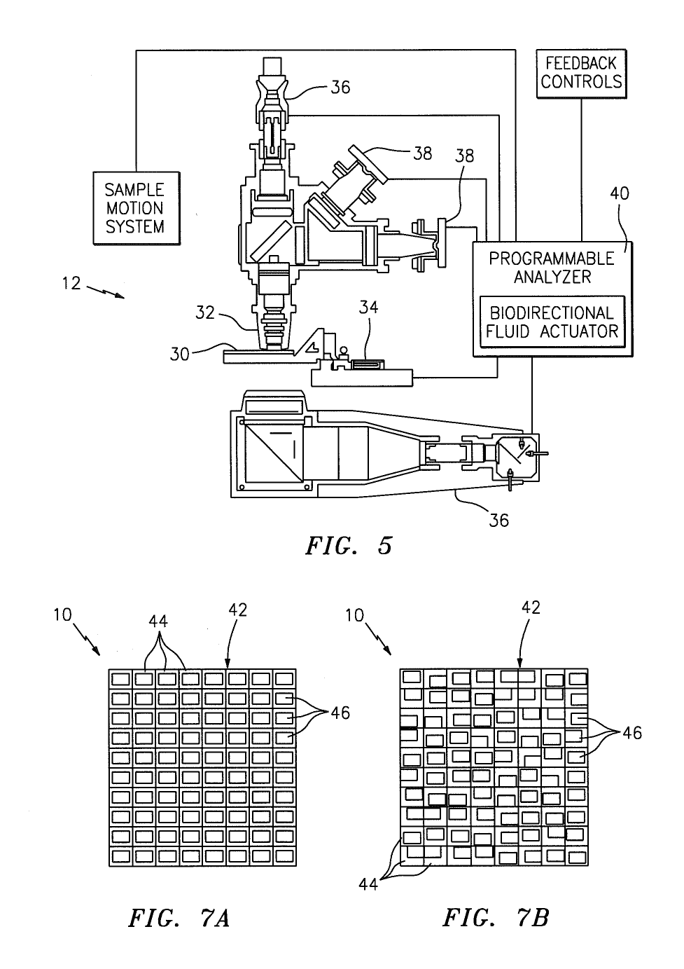

Referring to FIGS. 1 and 5, the present invention includes a method and an apparatus for analyzing a biological fluid sample (e.g., whole blood) quiescently residing within an analysis chamber, which analysis chamber 10 is configured to permit automated analysis of the sample by an analysis device 12. The sample quiescently residing within the chamber 10 is imaged, and the image of the sample is analyzed using the analysis device 12.

An illustration of a chamber 10 that can be used with the present invention is shown in FIGS. 1-4. The chamber 10 is formed by a first planar member 14, a second planar member 16, and typically has at least three separators 18 disposed between the planar members 14,16. At least one of the planar members 14,16 is transparent. The height 20 of the chamber 10 is typically such that sample residing within the chamber 10 will travel laterally within the chamber 10 via capillary forces. FIG. 4 shows a cross-section of the chamber 10, including the height 20 of the chamber 10 (e.g., Z-axis). FIG. 3 shows a top planar view of the chamber 10, illustrating the area of the chamber 10 (e.g., the X-Y plane). The lateral boundaries of the chamber 10 may be defined, for example, by glue lines 22 extending between the interior surfaces 24,26 of the planar members 14,16, or by lines of hydroscopic material disposed on a planar member surface that inhibit lateral travel there across.

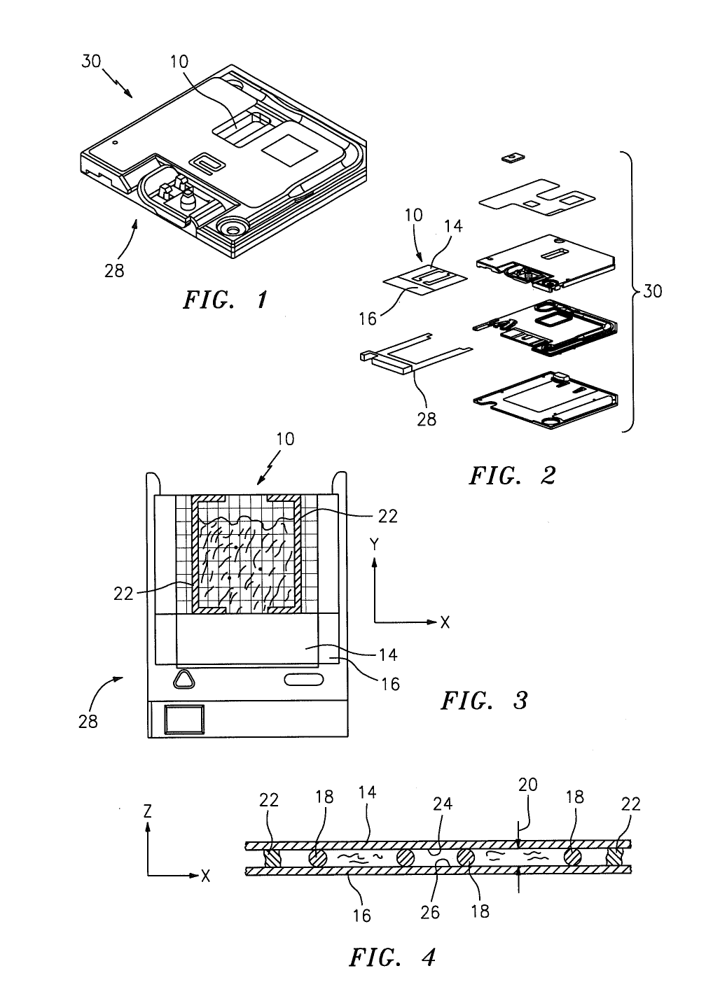

The present invention is not limited to use with any particular chamber embodiment. Examples of acceptable chambers are described in U.S. Pat. No. 7,850,916, and U.S. patent application Ser. Nos. 12/971,860; 13/341,618; and 13/594,439, each of which are incorporated herein by reference in its entirety. For purposes of this disclosure, the invention will be described as using the analysis chamber described in U.S. patent application Ser. No. 13/594,439. The analysis chamber 10 disclosed in the '114 application is mounted on a tray 28 that is removable from a cartridge 30. FIG. 1 shows the cartridge 30 in assembled form. FIG. 2 shows an exploded view of the cartridge 30, including the analysis chamber 10 and the tray 28. FIG. 3 is a top view of the analysis chamber 10 mounted on the tray 28, depicting a sample residing within the chamber 10. FIG. 4 is a diagrammatic cross-section of the chamber 10. The present invention is not limited, however, to use with the aforesaid chamber.

The analysis chamber 10 is typically sized to hold about 0.2 to 1.0 μl of sample, but the chamber 10 is not limited to any particular volume capacity, and the capacity can vary to suit the analysis application. The chamber 10 is operable to quiescently hold a liquid sample. The term “quiescent” is used to describe that the sample is deposited within the chamber 10 for analysis, and is not purposefully moved during the analysis. To the extent that motion is present within the blood sample, it will predominantly be due to Brownian motion of formed constituents within the blood sample, which motion is not disabling of the use of this invention.

Referring to FIG. 5, an automated analysis device 12 is shown that controls, processes, images, and analyzes the sample disposed within the cartridge 30. U.S. Pat. No. 6,866,823 and U.S. patent application Ser. Nos. 13/077,476 and 13/204,415 (each of which is hereby incorporated by reference in its entirety) disclose examples of analysis devices 12 that have optics and a processor for controlling, processing, and analyzing images of the sample, which devices can be modified according to the present invention as will be described below.

The analysis device 12 includes optics including at least one objective lens 32, a cartridge positioner 34, a sample illuminator(s) 36, an image dissector 38, and a programmable analyzer 40. The positioner 34 is adapted to selectively change the relative positions of the objective lens 32 and the analysis chamber 10. One or both of the optics (e.g., the objective lens) and the analysis chamber 10 are moveable relative to the other along all relevant axes (e.g., X, Y, and Z). Relative movement of the chamber 10 in the X-Y plane permits the optics to capture all fields of the sample residing within the chamber 10. Relative movement of the chamber 10 along the Z-axis permits change in the focal position of the optics relative to the sample height. The optics include hardware that enables the analysis device 12 to capture one or more low resolution images of the sample residing within the chamber 10, as well as one or more high resolution images of the sample within the chamber 10. Acceptable optical hardware capable of taking both low and high resolution images of the sample include embodiments that have a plurality of objective lenses (e.g., a high resolution objective lens and a low resolution objective lens) and embodiments wherein a single objective lens is used with one or more lenses that can be selectively moved into the optical path and are operable to change the resolution of the device. The present analysis device 12 is not limited to this exemplary optical hardware, however.

The sample illuminator 36 illuminates the sample using light at predetermined wavelengths. For example, the sample illuminator 36 can include an epi-fluorescence light source and a transmission light source. The transmission light source is operable to produce light at wavelengths associated with one or more of red, green, and blue light. The red light is typically produced in the range of about 600-700 nm, with red light at about 660 nm preferred. The green light is typically produced in the range of about 515-570 nm, with green light at about 540 nm preferred. The blue light is typically in the range of about 405-425 nm, with blue light at about 413 nm preferred. Light transmitted through the sample, or fluoresced from the sample, is captured using the image dissector, and a signal representative of the captured light is sent to the programmable analyzer, where it is processed into an image. The image is produced in a manner that permits the light transmittance or fluorescence intensity captured within the image to be determined on a per unit basis; e.g., “per unit basis” being an incremental unit of which the image of the sample can be dissected, such as a pixel.

An example of an acceptable image dissector 38 is a charge couple device (CCD) type image sensor that converts light passing through (or from) the sample into an electronic data format image. Complementary metal oxide semiconductors (“CMOS”) type image sensors are another example of an image sensor that can be used. The signals from the image dissector 38 provide information for each pixel of the image, which information includes, or can be derived to include, intensity, wavelength, and optical density. Intensity values are assigned an arbitrary scale of, for example, 0 units to 4095 units (“IVUs”). Optical density (“OD”) is a measure of the amount of light absorbed relative to the amount of light transmitted through a medium; e.g., the higher the “OD” value, the greater the amount of light absorbed during transmission. OD can be quantitatively described in optical density units (“ODU”) or fractions thereof; e.g., a MilliODU is a 1/1000^{th} of an ODU. One “ODU” decreases light intensity by 90%. “ODU” or “MilliODU” as a quantitative value can be used for images acquired or derived by transmission light.

The programmable analyzer 40 includes a central processing unit (CPU) and is in communication with the cartridge positioner 34, sample illuminator 36, and image dissector 38. The programmable analyzer 40 is adapted (e.g., programmed) to send and receive signals from one or more of the cartridge positioner 34, the sample illuminator 36, and an image dissector 38. For example, the analyzer 40 is adapted to: 1) send and receive signals from the cartridge positioner 34 to position the cartridge 30 and chamber 10 relative to one or more of the optics, illuminator, and image dissector; 2) send signals to the sample illuminator 36 to produce light at defined wavelengths (or alternatively at multiple wavelengths); and 3) send and receive signals from the image dissector 38 to capture light for defined periods of time. It should be noted that the functionality of the programmable analyzer may be implemented using hardware, software, firmware, or a combination thereof. A person skilled in the art would be able to program the processing unit to perform the functionality described herein without undue experimentation.

Referring to FIG. 6, under the present method the analysis device 12 is adapted to initially create one or more low resolution images of the sample quiescently disposed within the chamber 10 and subsequently to provide one or more high resolution images of the sample quiescently residing within the chamber 10. The images are subsequently communicated to the programmable analyzer 40 for one or more analyses of the sample based on the images of the sample. For purposes of performing analyses of substantially undiluted whole blood samples within a chamber 10 as described above, images having a resolution of greater than about 0.5 μm (micrometers) are adequate for performing the “low resolution” analyses described herein, and images having a resolution of less than about 0.5 μm are adequate for performing the “high resolution” analyses described herein. These values are examples of high and low resolutions that are useful for whole blood analyses (e.g., a complete blood count—CBC), and the present invention is not limited to them. A resolution higher than the 0.5 μm example given may be useful to provide additional accuracy and/or additional information; e.g., information that can be used to identify cell abnormalities and cell differentiations (e.g., IG, blasts, atypical lymphocytes, etc.).

The resolution of the low resolution image is adequate to identify certain characteristics of the image. For example, the low resolution image is adequate to permit identification of the boundaries of the sample within the chamber 10; e.g., lateral perimeter/sample interfaces (e.g., a glue line/sample interface, a hydrophobic line/sample interface, etc.), or a sample/air interface, etc. The low resolution image is also adequate to permit a volumetric determination of the sample quiescently residing within the chamber 10; e.g., with a known or determinable chamber height (the sample extends between the interior surfaces of the chamber planar surfaces) and the determined area of the sample, the volume of the sample can be determined. Many blood analysis parameters are volumetrically based, consequently being able to easily and quickly determine the volume is advantageous. The low resolution image is also adequate to identify WBCs, RBCs, or platelets within the sample, and in particular areas where WBCs, RBCs, and/or platelets congregate. The low resolution image provides sufficient information to permit a WBC or a platelet count. The low resolution image is also adequate to determine a hemoglobin concentration of the sample. The amount of information provided by the low resolution image is limited, however. The low resolution image typically does not provide enough information to permit a full WBC differential determination (e.g., a 5-part differential). The amount of information provided by the low resolution image is also inadequate for detailed RBC analyses such as a mean cell volume determination or other more detailed RBC indices.

The resolution of the high resolution image, in contrast, is adequate to provide additional information that is sufficient to enable additional analyses. For example, the high resolution image provides enough information to permit an accurate WBC differential determination. U.S. patent application Ser. No. 13/204,415, entitled “Method and Apparatus for Automated Whole Blood Sample Analyses from Microscopy Images”, which is hereby incorporated by reference in its entirety, discloses a methodology for performing a WBC differential on a whole blood sample. The high resolution image of the present method is adequate to enable determination of the features described in the aforesaid methodology, which features enable the identification of the specific type of any WBC identified within the low resolution image. Another analysis that can be performed using the high resolution image is described in U.S. patent application Ser. No. 13/051,705, entitled “Method and Apparatus for Determining at Least One Hemoglobin Related Parameter of a Whole Blood Sample”, which is hereby incorporated by reference in its entirety. The '705 application discloses a method for determining RBC indices including RBC cell volume (CV), mean cell volume (MCV), cell hemoglobin concentration (CHC), mean cell hemoglobin concentration (MCHC), and mean cell hemoglobin content (MCH), as well as their population statistics.

In one embodiment, the analysis device 12 is adapted to take a single image of the entire analysis chamber 10 at the low resolution level. Alternatively, a plurality of smaller area images can be subsequently combined to form the low resolution image of the analysis chamber 10. Although a single low resolution image is not required under the present invention, a single image is advantageous because typically it can be processed in less time. The single, or combined, low resolution image is subsequently communicated from the image dissector to the programmable analyzer where the content of the image is analyzed to establish the sample boundaries, WBC, RBC and/or platelet locations, WBC and/or platelet enumeration, RBC locations, sample volumetric determination, etc, as described above. The identification of WBCs within the low resolution image can be performed, for example, by epi-fluorescent analysis wherein the WBCs within the sample are stained with a fluorescent dye and the sample subjected to light at wavelengths that cause the dye to fluoresce. Epi-fluorescence can also be used to locate reticulocytes within the sample. Transmittance techniques can be used to locate RBCs within the low resolution image(s). The sample chamber 10 is also mapped to enable identification of the relative locations of the aforesaid constituents/features. The mapping is described hereinafter as a two-dimensional Cartesian grid map defining grid squares. The mapping is not limited to a two-dimensional Cartesian grid map, however, and may alternatively use any acceptable coordinate system (e.g., a polar coordinate system). The mapping also is not limited to an X-Y map; e.g., the map may include a Z-axis. The term “grid squares” is used to depict sub-areas defined by the map. The grid squares are not limited to any particular geometry, and are not required to have four equal length sides.

The ability of the present invention to use one or more low resolution images to locate certain constituents within the sample image is significant. Sample entering the analysis chamber typically distributes within the chamber via capillary action. During the sample distribution, it is often the case that sample constituents (e.g., WBCs, RBCs, platelets) do not uniformly distribute within the chamber. For example, upon the entire sample entering the chamber, WBCs often reside near the point of entry and RBCs often reside toward the leading edge of the sample (i.e., the edge opposite the point of entry). Consequently, imaging an area of the chamber to perform an analysis on a particular type of constituent (e.g., WBCs) where the constituent typically does not reside (e.g., the leading edge area) is not likely to provide substantial useful information, in contrast to an area of the chamber where the constituent typically resides (e.g., proximate the chamber entry area). In addition, the analysis chamber is typically configured to contain a volume of sample that is likely in all instances to include more than enough of each type of constituent that may be analyzed. Because of the substantial population differences between the various constituents (e.g., the number of RBCs in whole blood far exceeds the number of WBCs), however, that means that there is typically far greater numbers of constituent available for analysis than are statistically required for accuracy purposes. The ability of the present invention to use one or more low resolution images to locate a statistically adequate number of constituents without having to image the entire chamber provides significant advantage.

The number of high resolution images taken will likely vary depending upon information determined from the low resolution analysis, the particular type of analysis being performed, the particular resolution desired, and the manner in which chamber height focus (i.e., Z-axis focus) is acquired. Under the present invention, however, it is possible to provide the desired information using substantially fewer high resolution images than would be necessary to image the entire analysis chamber 10. As a result, the image processing time and therefore the time required to provide analysis results can be substantially reduced. For example, if less than the entire analysis chamber 10 is filled with sample, identification of the sample boundaries eliminates the need to acquire high resolution images of those grid squares where no sample resides. In a sample chamber 10 having a width of about nine millimeters (9 mm) and a length of about fourteen millimeters (14 mm), a total of 80-100 high resolution images is typically adequate to image the entire analysis chamber 10. If the sample only fills “X” percentage of the entire chamber 10, then the total number of images (e.g., 80-100) can initially be reduced by “X” percent. Furthermore, if the analysis at hand only requires information from particular constituents within the sample, then only those grid squares containing the particular constituents need be imaged at a high resolution; e.g., if the analysis only requires a WBC enumeration, then high resolution images of those grid squares containing WBCs need be imaged. As a result, the number of high resolution images can initially be decreased by “X” percent and then further reduced to only the number of images necessary to capture the desired constituents. The amount of image data collected and the concomitant processing time is advantageously reduced.

Another aspect of the present invention involves analyzing the low resolution images to determine certain types of information that are available at low resolution, and subsequently using that information in the determination of whether additional analyses are required, which analyses require high resolution imaging. If for example, the information available at low resolution indicates that the sample appears normal without any indicator of a health issue, then the analysis of the sample may be terminated. On the other hand, if the information available at low resolution indicates that the sample appears abnormal, then additional analyses may be performed on the sample, including those that require high resolution imaging. Performing the low resolution “screening” can prevent the time and cost of performing unnecessary analyses.

Another aspect of the present invention includes an additional technique for processing substantial numbers of images; e.g., a substantial number of high resolution images, each at different Cartesian grid coordinates. In those instances where a large number of images are taken at different Cartesian grid coordinates, and if the collective image must capture the entire sample quiescently residing in the chamber 10, it is necessary to utilize a precise cartridge positioner, one that can prevent overlap between adjacent images and/or un-imaged spaces between adjacent captured images. A cartridge positioner with that level of accuracy can add significantly to the cost of the analysis device 12, and can also slow the processing time. In addition, imaging 100% of the sample within the chamber 10 also increases the image processing time, image data storage requirements, and slows communication speeds when images are communicated out from the analysis device 12.

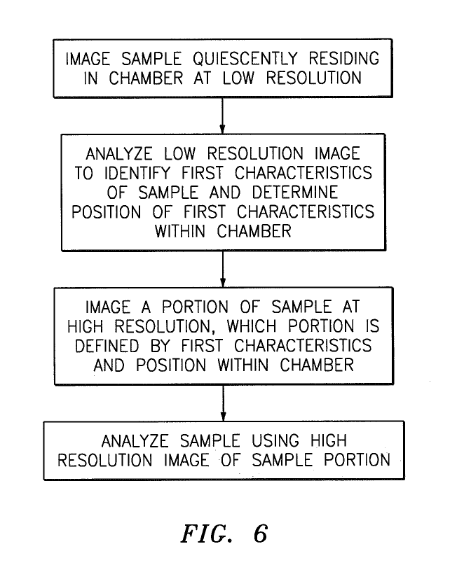

According to this aspect of the present invention, an image (e.g., a high-resolution image) is acquired within each grid square, which image captures an area that is less than the entire area of the grid square; i.e., if the area defined by a grid square is equal to “A”, then the area captured by the image associated with that grid square under this aspect is less than “A”. To illustrate, FIGS. 7A and 7B diagrammatically illustrate a Cartesian grid map 42 applied to an analysis chamber 10. In FIG. 7A, the image portion 46 of each grid square 44 is less than the entire grid square 44, but is centered within the respective grid square 44. In FIG. 7B, the image portion 46 of each grid square 44 is also less than the entire grid square 44, but is randomly positioned within the respective grid square 44. The term “randomly positioned” is used herein to reflect that the tolerance of the cartridge positioner relative to the dimensions of the grid square 44 is such that the actual position of the image portion can randomly be found anywhere within the grid square 44 and still be within the defined tolerance limits of the cartridge positioner. Hence, a cartridge positioner with a lower positional accuracy can be used to take the “randomly positioned” image portions disposed within the grids 44 shown in FIG. 7B.

The decreased size of the imaged portion within each grid square 44 does collectively result in less than 100% of the sample being imaged and available for analysis. The maximum amount of the decrease in sample image that is acceptable (i.e., the decreased sample image still yields acceptable analysis accuracy) will likely depend on the analysis at hand. To assess the accuracies of analyses performed on partial collective sample images versus complete collective sample images, a couple of analyses (hemoglobin content and WBC count) were performed on a dataset of images of substantially undiluted whole blood samples disposed within a chamber 10. In the hemoglobin analyses, the image portions were each decreased by an amount that resulted in a collective partial sample image being about 52% of the entire collective sample image. The partial image analyses results agreed with the complete image analyses more than 95% of the time. In the WBC count analyses, the image portions were each decreased by an amount that resulted in a collective partial sample image being about 67% of the entire collective sample image. The partial image analyses results agreed with the complete image analyses more than 97% of the time. Consequently, this aspect of the present invention provides another technique that can be used to decrease the amount of time required to acquire sample images for subsequent analysis, with a relatively low change in accuracy. The analyses described above are examples of investigations that can provide objective data to assess the merit of a technique such as that described. The present methodology and apparatus for rapid imaging of biologic fluid samples, relatively speaking, is not limited to use with any particular type of analysis.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed herein as the best mode contemplated for carrying out this invention.

What is claimed is:

1. A method for analyzing a biologic fluid sample, comprising the steps of:
disposing the biologic fluid sample within a chamber adapted to quiescently hold the biologic fluid sample;
imaging the biologic fluid sample at a first resolution, and producing first image signals representative of a low resolution image of the sample;
analyzing the first image signals to identify one or more first characteristics of the sample, and determining a position of each first characteristic within the chamber using a map of the chamber;
imaging a portion of the biologic fluid sample at a second resolution and producing second image signals representative of a high resolution image of the sample, which portion of the biologic fluid sample is determined using the one or more first characteristics and the map, and wherein the second resolution is greater than the first resolution; and
analyzing the biologic fluid sample using the second image signals.

2. The method of claim 1, wherein the chamber includes a first planar member and a second planar member, and the first characteristics of the sample include lateral perimeters of the sample quiescently residing within the chamber.

3. The method of claim 2, wherein the lateral perimeters of the sample within the chamber include a sample-glue line interface, which glue line extends between a surface of the first planar member and a surface of the second planar member.

4. The method of claim 2, wherein the lateral perimeters of the sample within the chamber include a sample-hydrophobic line interface, which hydrophobic line is disposed on one or both of the first planar member and the second planar member.

5. The method of claim 2, wherein the lateral perimeters of the sample within the chamber include a sample-air interface.

6. The method of claim 2, wherein the step of imaging a portion of the biologic fluid sample at a second resolution is applied to the portion of the biologic fluid sample surrounded by the lateral perimeters.

7. The method of claim 1, wherein the step of analyzing the first image signals to identify one or more first characteristics of the sample includes identifying WBCs within the sample.

8. The method of claim 7, wherein the step of analyzing the first image signals to identify one or more first characteristics of the sample includes locating the identified WBCs within the sample.

9. The method of claim 7, wherein the step of analyzing the first image signals includes performing a WBC count.

10. The method of claim 1, wherein the imaging of the biologic fluid sample at a first resolution includes taking a single image of all sample residing within the chamber.

11. The method of claim 1, wherein the map of the portion of the sample defines a plurality of grid squares, and the step of imaging the biologic fluid sample at the second resolution includes taking an image of the sample aligned with each grid square of the sample portion, and collectively forming the high resolution image of the sample from the grid square images of the sample portion.

12. The method of claim 11, wherein each grid square has an area, and the sample image aligned with each grid square has an area that is less than the area of each grid square.

13. The method of claim 1, wherein the map of the chamber is one of the following two-dimensional coordinate systems: Cartesian coordinate system, or polar coordinate system.

14. An apparatus for analyzing a biologic fluid sample quiescently disposed within an analysis chamber, the apparatus comprising:
an objective lens assembly operable to image the biologic fluid sample at a first resolution and a second resolution, which second resolution is greater than the first resolution;
at least one image dissector;
a processor adapted to analyze first image signals produced by the image dissector with the objective lens at the first resolution, to identify one or more first characteristics of the sample, and to determine a position of each first characteristic within the chamber using a map of the chamber, and the processor is further adapted to create an image of a portion of the biologic fluid sample at the second resolution and to produce second image signals representative thereof, which portion of the biologic fluid sample is determined using the one or more first characteristics and the map, and the processor is further adapted to analyze the biologic fluid sample using the second image signals.

15. The apparatus of claim 14, wherein the chamber includes a first planar member and a second planar member, and the first characteristics of the sample include lateral perimeters of the sample quiescently residing within the chamber.

16. The apparatus of claim 15, wherein the lateral perimeters of the sample within the chamber include a sample-glue line interface, which glue line extends between a surface of the first planar member and a surface of the second planar member.

17. The apparatus of claim 15, wherein the lateral perimeters of the sample within the chamber include a sample-hydrophobic line interface, which hydrophobic line is disposed on one or both of the first planar member and the second planar member.

18. The apparatus of claim 15, wherein the lateral perimeters of the sample within the chamber include a sample-air interface.

19. The apparatus of claim 15, wherein the processor is adapted to image a portion of the biologic fluid sample at a second resolution, which portion of biologic fluid sample is surrounded by the lateral perimeters.

20. The apparatus of claim 14, wherein the processor is adapted to analyze the first image signals to identify WBCs within the sample.

21. The apparatus of claim 14, wherein the processor is adapted to analyze the first image signals and perform a WBC count.

22. The apparatus of claim 14, wherein the objective lens assembly is operable to take a single image of all sample residing within the chamber at the first resolution.

23. The apparatus of claim 14, wherein the map of the portion of the sample defines a plurality of grid squares, and the processor is adapted to image the biologic fluid sample at the second resolution, including taking an image of the sample aligned with each grid square of the sample portion, and to collectively form the high resolution image of the sample from the grid square images of the sample portion.

24. The apparatus of claim 23, wherein each grid square has an area, and the sample image aligned with each grid square has an area that is less than the area of each grid square.

25. The apparatus of claim 14, wherein the map of the chamber is one of the following two-dimensional coordinate systems: Cartesian coordinate system, or polar coordinate system.

26. A method for imaging a biologic fluid sample, comprising the steps of:
disposing the biologic fluid sample within a chamber adapted to quiescently hold the biologic fluid sample, which sample fills an area within the chamber;
mapping the chamber, which map defines a plurality of grid squares, each grid square having an area;
imaging portions of the biologic fluid sample, each image portion aligned with a different grid square of the map, and each image portion having an area, and which image portions together form a collective image of the sample residing within the chamber, and wherein the collective area of the image portions has an area that is less than the area filled by the sample residing within the chamber.

27. The method of claim 26, wherein the map of the chamber is one of the following two-dimensional coordinate systems: Cartesian coordinate system, or polar coordinate system.
